(19) United States
(12) Patent Application Publication
(10) Pub. No.: US 2011/0081706 A1
(43) Pub. Date: Apr. 7, 2011

(54) Method and system for efficient harvesting of microalgae and cyanobacteria

(75) Inventors: Ami Schlesinger, Kibbutz Maagan Michael (IL); Doron Eisenstadt, Haifa (IL); Shai Einbinder, Hofit (IL); Jonathan Gressel, (US)

(73) Assignee: TransAlgae Ltd, Rehovot (IL)

(21) Appl. No.: 12/924,460

(22) Filed: Sep. 28, 2010

Related U.S. Application Data
(60) Provisional application No. 61/278,205, filed on Oct. 2, 2009.

(51) Int. Cl. C12N 1/12; C12N 1/20

(52) U.S. Cl. 435/252.1; 435/257.1; 435/257.6

(57) ABSTRACT

The high-speed centrifugation heretofore required for harvesting micro algae and cyanobacteria cultured for biofuels and other co-products is a major cost constraint. Mixing algae/cyanobacteria at high-density culture with far less alkali than previously assumed is sufficient to flocculate the cells. The amount of flocculant required is a function of the logarithm of cell density, and is not a linear function of cell density as had been thought. The least expensive alkali treatments are with slaked limestone or dolomite (calcium hydroxide and magnesium hydroxides). Further water can be removed from the floc by sedimentation, low speed centrifugation, dissolved air flotation or filtration, prior to further processing to separate oil from valuable co-products.

### PRIORITY

This application claims priority of U.S. provisional application No. 61/278,205 filed on Oct. 2, 2009.

### FIELD OF THE INVENTION

This invention relates to the field of harvesting microalgae and cyanobacteria. More specifically this invention relates to a method and system of inexpensively and efficiently concentrating microalgae and cyanobacteria from culture media prior to extraction of oil and other valuable products.

### BACKGROUND OF THE INVENTION

The use of renewable energy sources is becoming increasingly necessary due to today's high energy prices and impacts of climate change. Microalgae and cyanobacteria are very efficient solar energy converters when compared with land plants, and in addition to fast biomass production, they can produce a great variety of metabolites. Some species are especially valuable as they can be harnessed for oil production. In addition, biofuel and feed production in marine algae do not entail a decrease in food production, as does production of biofuels from common food crops such as maize, soybean or oilseed rape.

The inefficiency and high cost of centrifugation required to separate the microalgae/cyanobacteria at the time of harvest imposes a major challenge to the use of microalgae and cyanobacteria for production of oils for biofuels and for other uses. Alternatively, methods of flocculation have been tried in the past, but these either utilize toxic flocculants (such as aluminum salts), expensive flocculants (chitosans) or high levels of alkali, ostensibly needed to counter the surface charge on each cell (to reach pH 10-11, in various studies).

### Flocculation

Flocculation has been considered too expensive as a mechanism of harvesting algae for low cost bulk products. Sayres (2009) has updated the cost of centrifugation made by Becker (1994) to a present cost of $2.40/kg algae harvested, and claims that “harvesting by flocculation or flotation is only marginally less expensive (than centrifugation)”. Various methods have been tested in the past to induce flocculation, many borrowed from sewage treatment technologies, where it is necessary to precipitate small suspended solids from solutions where the solutions are colloidal and thus not allowing rapid sedimentation, and/or the particles have a similar specific gravity as the solution. They are all based on the premise that the small particles (algae and cyanobacteria in the present case) have a surface charge that repels them from one another. Flocculants remove this surface charge, allowing the particles to stick to each other, generating flocs. It has been assumed that there is a direct, linear stoichiometric relationship between number of cells and the amount of flocculant required, and thus costs per ton of algae would remain constant, and a function of flocculant cost.

It must be kept in mind that sewage flocculation has a very different economic end point from algal flocculation. The floc from sewage ends up in a landfill or at best as a compost-like material for agriculture. The flocculant for sewage can add bulk, and the flocculant may even have a modicum of toxicity to other organisms. With algae, the end products are harvested oil, animal feed, various secondary metabolites and/or other products to be marketed. Thus inedible bulk or toxic, non-nutritious or otherwise deleterious flocculants that can be used with sewage are inadequate for the algae industry. As discussed below, there are some physical methods of countering the surface charge of algae and cyanobacteria, but they are only cost-effective with freshwater species. Due to the shortage of freshwater on this planet much of the culturing of algae will utilize sea- or brackish water. Thus, any flocculation procedure must be compatible with saline water. The classic extrapolation from sewage treatment was the addition of clay to freshwater algae, to cause a mutual flocculation (Avnimelech et al., 1982). The cyanobacterium Anabaena required almost twice as much clay as algae to flocculate, and even then, a base had to be added to achieve flocculation (e.g. 20 mM NaOH (i.e. a pH value of more than 12). Various cationic organic polymers are typically used for sewage treatment, but the high salinity of the seawater inhibits flocculation with such polyelectrolytes. Maximum filterability was obtained at the point of charge neutralisation where the algal cells formed aggregates (Bernhardt and Clasen, 1994). At high ionic strength of seawater, these polymers shrink to their smallest dimensions, and fail to bridge between algal cells (Bilanovic et al., 1988). Polyelectrolytes used for flocculation such, as polyacrylamide would be inappropriate in animal feeds. The natural polymer, chitosan, derived from shrimp exoskeletons (“shells”) has been used for harvesting algae grown in both freshwater and seawater (Nigam et al., 1980; Lavoie & de la Nofie, 1983; Morales et al., 1985) but is expensive and adds bulk.

Alum (hydrated aluminum potassium sulfate) and other aluminum salts are widely used as flocculants for sewage and algae but are undesirable for animal feed unless the aluminum is removed (e.g. U.S. Pat. No. 4,680,314). Alkaline iron III hydroxide may also be used as a flocculant.

Ultrasound and other physical treatments have been used to harvest algae or some of their products. For example, 80 kHz ultrasound waves were used to acoustically cavitate the gas vacuoles that control the floating of the cyanobacterium Spirulina in water together with the flocculant polychlorinated aluminum, increasing the efficiency of flocculation by the flocculant alone from 65% to above 90% (Zhang et al. 1996). Ultra-sound with decane has been used to repeatedly and non-lethally release part of the oil from algae, precluding the need to harvest the algae (Sayre, US Patent Application 2009/0181438). This technique is suitable for algae cultured for a single purpose, such as oil, but does not provide other co-products. The energy required and the suitability for seawater environments has not been reported. A company, OriginOil also claims to be using high ultrasonic intensity with water and “special catalysts” to crack the algae membrane to facilitate extracting its oil content. They describe this “quantum fracturing” as using a combination of pulsed electromagnetic fields and lowered pH using CO₂ to break the cell walls, with lipid floating and residual cell mass flocculating and sedimenting “in less than an hour”. No information is presented on whether part of the soluble cell contents leak out during this process, nor is there any information regarding the energy requirements and costs for this process.

Electrolytic flocculation (electrolysis) needs relatively little electricity to flocculate freshwater micro-algae from a suspension and subsequently float the algal flocs (Poelman et al., 1997), but the high conductivity of seawater renders this unpractical for marine algae and cyanobacteria. Similarly, electrolysis integrating electroflotation and electroflocculation was achieved by using a polyvalent aluminum alloy anode for flocculant generation and an inactive titanium alloy cathode generating the gas bubbles for flotation (Alfafara et al., 2002). This system would also not be applicable for the high conductivity of seawater.

A group at the University of Texas Center for Electromechanics (Hebner, Werst and Davey) are using electromechanical charges for oil extraction from algae. They calculated the operational costs for extraction from fresh water cultivated algae to be acceptable $0.04/gal algae oil, the electromechanical extraction of algae cultured in brackish water to be $0.26/gal algae oil, and according to them it would be much more expensive in seawater.

It was realized by various groups, that algae and cyanobacteria could be flocculated by high pH. For example, the best flocculation of the freshwater green alga Botryococcus braunii could only be achieved after two weeks in batch culture. The high pH value (pH 11) needed was achieved with NaOH, which was more effective for flocculation than treatment with aluminum sulfate or a bacterial derived substituted polysaccharide flocculant, “Pestan”, until the third week of incubation (Lee et al 1996).

Calcium carbonate formed due to reaction of calcium hydroxide with the dissolved carbon dioxide in the water (H₂CO₃) can precipitate out of solution at a pH range of 9.1-9.5, and act through a ‘sweep coagulation’ mechanism to entrap suspended and colloidal particles. It also acts as a ‘weighting agent’ by increasing the density of the settling particles, thereby enhancing their settlement (Leentvar and Rebhun, 1982). It was discovered long ago that Mg(OH)₂ and Ca(OH)₂ flocculate algal suspensions (Folkman and Wachs 1973). It was theorized that pH plays an important role in the process. Particles begin to form at pH 10, and the flocculation only completed at pH 11. No flocculation occurred up to pH 10. Magnesium hydroxide forms a gelatinous precipitate, which served as an efficient coagulant and flocculation aid above pH 11 (Vrale, 1978), yet Mg(OH)₂ solutions can only reach pH 9.6, and a stronger base is required in addition to achieve the high pH required for the process. Thus, for the treatment process to operate efficiently, either Ca(OH)₂ or NaOH were used to increase the pH to pH 11.0-11.5 (>500 μM alkali) (Lee et al., 1998; Dziubek and Kowal, 1989; Semarjian and Ayoub 2003). The Mg(OH)₂ precipitate has a large adsorptive surface area and a positive superficial charge, which attracts the negatively charged colloidal particles, including the CaCO₃ flocs, thus inducing adsorption and agglomeration. This explains the significant efficiency achieved when Mg(OH)₂ is precipitated (Semarjian and Ayoub 2003).

Algal pastes resulting from high pH lime treatment possess superior thickening and dewatering characteristics and are suitable for filter pressing at lower cost than pastes generated from ferric chloride or alum (Semarjian and Ayoub, 2003).

Most studies described above (and many others: e.g.; Elmaleh et al. 1991, 1996, Yahi et al. 1994); McCausland et al. 1999. [pH 11.5-12]; Knuckney et al., 2006 [pH 10-10.6 non-ionic polymer Magnafloc LT-25]; U.S. Pat. No. 3,431,200 [1-2 g/l CaCl₂+0.5-1 g/l NaOH]; U.S. Pat. No. 3,780,471 [pH 11.4 with Ca and Mg]; Ayoub and Koopman, 1986 [pH>10-10.5], Banchemain and Grizeau 1999 [pH 10.2]) with calcium and magnesium used very high pH values. One study achieved good flocculation with calcium at a lower pH (8.5-9) by having large amounts of orthophosphate in the medium (Sukenik and Shelef 1984). They posited that the calcium phosphate precipitate acted as the flocculating agent. Even though their pH was low, they had to use 1.5-2.5 mM calcium to achieve flocculation. Similarly, 0.4-1.3 mM calcium hydroxide was needed to flocculate the diatom Phaeodactylum sp. (Veloso et al., 1991), and 1.3 mM was required for two other diatoms and Tetraselmis sp., whereas Isochrysis sp. could not be flocculated (Millamena et al. 1990). It has been assumed that flocculation works by removing the negative surface charges on the algal cells such that they no longer repel each other. If that were the case, it has been logically expected that the greater the concentration of algae in a culture, the more alkali needed to titrate the surface charges (Sayre 2009), as has been found in water treatment with alkali flocculation (Henderson et al., 2008a,b). The opposite was found with chitosan as the flocculant (Divakaran and Pillai, 2002). With higher algal densities, it is statistically “easier”: for this polymeric polyelectrolyte to “bridge” (cross link) between cells, forming aggregates. Such bridging is not expected with small molecular weight flocculants that do not have a large enough cross section to bridge.

Once achieved, a floc can be removed by dissolved air floatation (Levin et al., 1962; U.S. Pat. No. 3,780,471; Henderson et al., 2008c), or by sedimentation. Efforts to develop a belt filtration system by AlgaeVenture Systems have been successful for the larger single-celled algae, but have not yet been successful with the smaller microalgae (Jim Cook, AlgaeVenture Systems, 30 Jun. 2009, personal communication).

Accordingly, there is a need for an efficient and low cost method to harvest algae and cyanobacteria. There is a need for a system that would allow harvesting of fresh water and of salt-water species. Moreover, there is an unmet need for a method to harvest larger as well as smaller cell sized species. And yet there is an unmet need for a method that could be safely used for harvesting algae and cyanobacteria for any purposes, including use as feed, requiring that non-toxic materials be used. The present invention provides a novel, highly economical solution to these needs, which was contrary to the data and predictions in the scientific and technical literature.

### SUMMARY OF THE INVENTION

Accordingly, the invention described here provides a means to produce harvested algal or cyanobacterial paste at much lower energy and monetary costs than other currently available methods. The paste facilitates a lower cost of extraction of oil, proteins, and other co-products of value, or for direct processing as feed. The method according to this invention also provides production of spent material after extraction of primary products that can be used as an additive to animal feed directly, or with lower drying costs due to lower water content.

The method according to this invention utilizes alkali of which calcium and/or magnesium hydroxides are the least expensive alkaline materials available. The alkaline flocculants are used at more than a tenfold lower level of alkali than has been used by others, and is counterintuitive to the accepted wisdom that there is a direct linear relationship between number of cells to be flocculated and the amount of flocculant required. Instead, we found that the amount of flocculant required is directly related to the logarithm of cell density, i.e., the denser the cell suspension, the less flocculant needed per cell. The rapid and near complete flocculation, which removes up to 90% of external water in as little as 15 minutes, resulting in a floc (the precipitate that comes out of solution during the process of flocculation) that can be further concentrated by rapid and low cost sedimentation, or by filtration.

In one embodiment, the amount of alkali used can be further reduced by depleting the carbon dioxide from the growth medium prior to flocculation.

In yet another embodiment, the floc can be further concentrated by dissolved air flotation and cells in the floc are then “skimmed” from the top.

In another embodiment, the floc can be further concentrated by filtration.

In another embodiment, the floc can be further concentrated by low g-force centrifugation.

In yet another embodiment, the floc can be further concentrated and dewatered by continuous belt filtration and dewatering process.

In a further embodiment hydrodynamic separation in spiral separators (e.g. see US Patent Application publication US20090114601; US20090050538; and US20080128331), can be used as a pre-concentration system before flocculation to further reduce the amount of flocculant required, and/or following the addition of flocculant to get further fast water removal.

### SHORT DESCRIPTION OF THE DRAWINGS

FIG. 1 is a schematic presentation of the process according to this disclosure to pretreat, culture, and then harvest algae or cyanobacteria for various purposes.

FIGS. 2A and B. The amount of Ca(OH)₂ needed to induce flocculation is not the direct linear function predicted by theory (triangular symbols), but is a logarithmic function (note horizontal axis), and much less base is required than predicted by earlier conventional wisdom. Empiric results plotted for three algal cultures of Nannochloropsis salina (diamond symbols) and the expected linear plot (triangles). The R-squared value (R²=0.9, p<0.05) of the logarithmic regression indicates that approximately 90 percent of the variation in the flocculation activity of Ca(OH)₂ can be explained by the logarithm of N. salina cell density. B. Cells of Nannochloris sp. prior and post flocculation with CaOH (15%). Culture cell density was approx. 3.1×10⁸ cells/mL, with an average cell diameter of ˜2 μm. Culture pH was 7.1, and flocculation occurred at pH 9.8.

FIG. 3 Flocculation pH value (pH_{f}) increases according to the logarithm of cell density of Nannochloropsis salina (diamond symbols) and Isochrysis sp. CS-177 (square symbols). Each series represents three algal cultures. The R-squared value (R²=0.68, p<0.05) of the logarithmic regression indicates that approximately 68 percent of the variation in the pH_{f} of N. salina can be explained by the logarithm of culture density. The R-squared value (R²=0.94, p<0.05) of the logarithmic regression indicates that approximately 94 percent of the variation in the flocculation activity of Isochrysis sp. can be explained by the culture density. Both the pH values of flocculation and the amount of base per cell of these dense cultures is much lower than those reported in the literature.

FIG. 4 Algae culture pH does not affect flocculation pH value. Effect of Nannochloropsis salina culture pH on the pH value at flocculation. Values are averages of three replicates. Error bars depict standard errors. Culture pH does not significantly affect flocculation pH (t-test, N=6, p>0.1).

FIG. 5 The pH of algal culture media just prior to flocculation governs the amount Ca(OH)₂ needed to induce flocculation. Values are averages of three replicates. Error bars depict standard errors. Algae media pH significantly affected the amount of Ca(OH)₂ additive needed to induce flocculation of Nannochloropsis salina (t-test, N=6, p<0.05). The higher pH can be obtained by stopping the exogenous supply of carbon dioxide in the light so that it becomes photosynthetically depleted prior to flocculation.

FIG. 6 Efficiency of Nannochloropsis salina flocculation increases with cell density. Squares indicate the amount of natural sedimentation of the unflocculated controls, diamond markers indicate the amount sedimented after flocculation. The R-squared value (R²=0.89, p<0.05) of the logarithmic regression of the treatment values indicates that approximately 90 percent of the variation in the flocculation activity can be explained by the culture density.

FIG. 7 Efficiency of Isochrysis sp. CS-177 flocculation increases with cell density. Squares indicate the amount of natural sedimentation of the unflocculated controls, diamond markers indicate the amount sedimented after flocculation. The R-squared value (R²=0.83, p<0.05) of the logarithmic regression of the treatment values indicates that approximately 80 percent of the variation in the flocculation activity can be explained by the culture density.

FIG. 8 Slightly but significantly greater efficiency of flocculation of log vs. stationary phase cultures. Values are averages of three replicates. Error bars depict standard errors. Growth phase significantly affected the percent of flocculation in algae cultures. The efficacy of flocculation was higher in cultures of Nannochloropsis salina than of cultures of Isochrysis sp. (two-way ANOVA, N=6, p<0.05).

FIG. 9 Less flocculant added to induce flocculation in stationary phase cultures. Values are averages of three replicates. Error bars depict standard errors. Growth phase significantly affected the amount of Ca(OH)₂ supplement needed to induce flocculation. Values are averages of three replicates. Error bars depict standard errors. Relative stationary vs log efficacy of flocculant was higher in cultures of Isochrysis sp. than in cultures of Nannochloropsis salina (two-way ANOVA, N=6, p<0.05).

FIG. 10 Flocculation facilitates further concentration of Nannochloropsis oculata by low speed (50×g) centrifugation (A) and enhances the cell concentration factor (B).

(A) Ca(OH)₂ flocculated samples reached significantly higher concentrations than un-flocculated controls, 5 minute centrifugation reached higher concentrations than 1 minute but were not significantly lower than 10 minutes (two-way ANOVA, N=6, p<0.05). Values are averages of three replicates. Error bars depict standard errors.
(B) Flocculation enhances concentration factor of N oculata cells attained by centrifugation at 50×g. Flocculated cells have significantly higher concentration factors (concentration factor=final cell concentration/initial cell concentration) than control unflocculated samples. 5-minute centrifugation reached higher concentration factors than 1-minute but were not significantly lower than 10-minute centrifugation (two-way ANOVA, N=6, p<0.05). Values are averages of three replicates. Error bars depict standard errors.

FIG. 11 Hardened filter paper effectively filters flocculated algae cells. Values are averages of two repetitions. Filtration efficiency was expressed as 1−(OD_{(f)}/OD_{(c)}). Whatman 50 paper removed both flocculated and suspended algal cells. Whatman 54 retained over 90% of the flocculated cells and removes 50% of the suspended cells.

FIG. 12 Post flocculant biological related precipitations of phosphorous (P) and iron (Fe). Addition of alkaline flocculants to mineral fertilizer (Mor) enriched seawater (SW) does not precipitate phosphorous (P) and slightly precipitates some iron (Fe). Much of the difference between seawater with added minerals and flocculated seawater with added minerals can be accounted for by cellular assimilation of iron and phosphorus.

FIG. 13 Post flocculant biological related precipitation of potassium (K) and magnesium (Mg) in the different treatments. Phosphorous (P) and iron (Fe) are presented in FIG. 12. Addition of flocculant to (SW) does not affect concentrations of phosphorous (P), iron (Fe) or magnesium (Mg). Potassium (K) levels are unaffected by flocculation with NaOH or Ca(OH)₂. KOH based flocculation naturally increased K concentrations. Addition of flocculant to mineral fertilizer (Mor) enriched seawater algae cultures reduce P and Fe concentrations to SW values while K and Mg concentrations were unaffected.

### DETAILED DESCRIPTION OF THE INVENTION

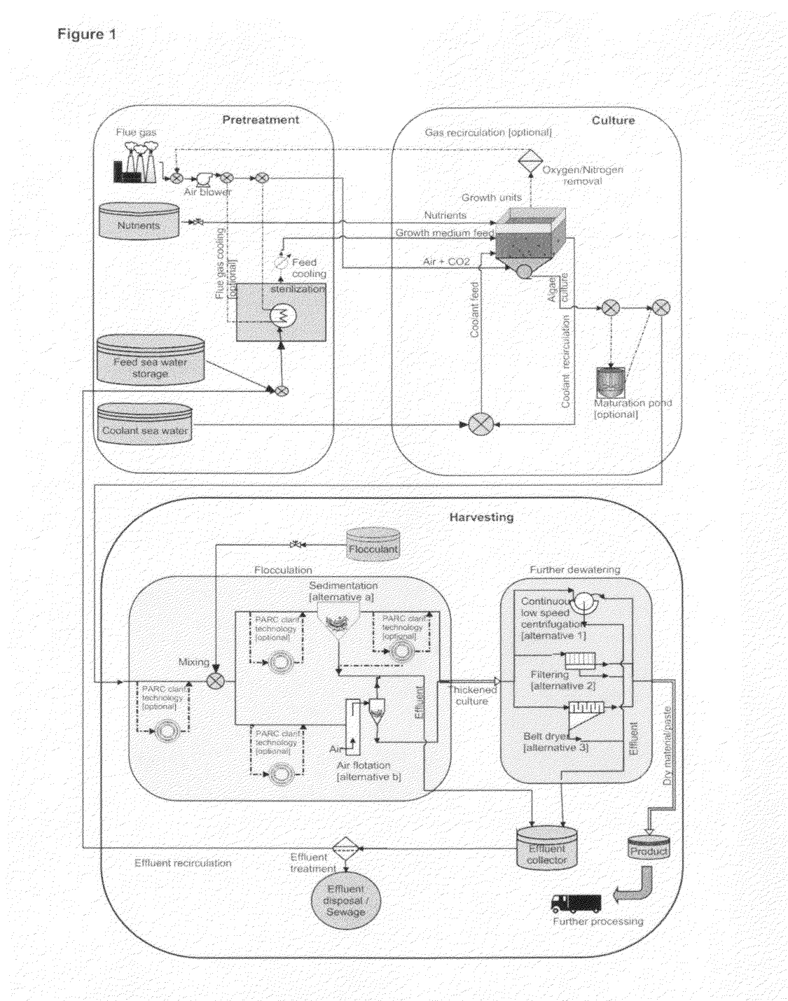

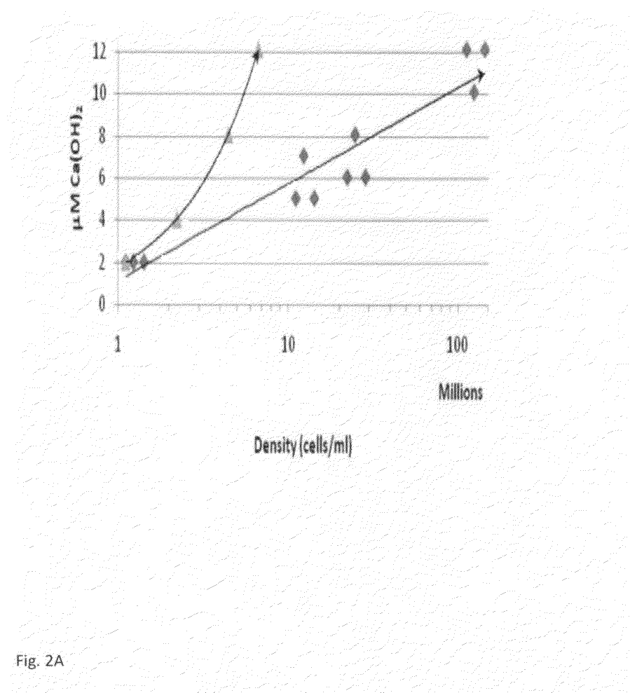

This invention relates in general to concentrating microalgae and cyanobacteria from culture media by flocculation as part of the harvesting process, by agglomerating them using alkali at-least 10-fold lower alkali concentrations than had been known to flocculate algae and cyanobacteria, so that the flocculated cells can easily be harvested by sedimentation or flotation, coupled with light centrifugation or filtration as outlined in FIG. 1. This brings enormous improvement over the energy-inefficient high-speed centrifugation systems, or the use of expensive and/or toxic flocculants, or high pH flocculation presently used in sewage and water treatments. The technology of this disclosure uses less Ca(OH)₂ to achieve flocculation than previously known technologies, and at current U.S. pricing would cost about $3.50 per ton of dry weight at a culture density of 10⁸ cells/ml, and $7.50 per ton at a culture density of 10⁷ cells/ml, at 2010 prices for lime (vs. the more than $2,000/ton predicted by Sayres (2009) by linear extrapolation). These base-flocculated algae can then be used as aquaculture feeds (Knuckney et al., 2006).

Microalgae and cyanobacteria are cultured using photosynthesis as a source of fixed carbon for growth and cell division. The cells are either cultured in sophisticated tube, cylinder, or flat-plate reactors or “raceway ponds” which are shallow ponds where the medium is continually moved using paddle wheels, or in other open or enclosed systems that give high densities of algae needed to render this non-linear, exponentially functioned flocculation technology viable. During the rapid division of algal cells their oil content is typically relatively low. Once ponds or bioreactors achieve maximum working densities, the cells can be removed to maturation reactors where cells partition photosynthates into oil bodies as the essential minerals as the media become depleted, or the cells can be harvested during late logarithmic growth.

Genetic modification of the starch metabolism of microalgae and cyanobacteria to reduce cell starch content increases the oil content and/or reduces specific high content proteins such as RUBISCO (ribulose-1,5-bisphosphate carboxylase/oxygenase) and increase the concentration of more valuable proteins. Moreover, the algae and cyanobacteria may have modified starch metabolism. It is especially useful for the algae to have reduced chlorophyll antenna components, as this may allow algae to grow to higher densities with the same level of light penetration. The method and the algal cells possessing such modified characters are disclosed and claimed in other patent applications of our research group. The currently claimed harvesting system is preferably used to harvest oil from such modified algae, but this harvesting system can be used with any algae or cyanobacteria at high densities used for most purposes.

When the desired oil or other product content of the algal or cyanobacteria cells is achieved, sufficient alkali is mixed into the culture until there is flocculation. The pH at which this occurs varies among species, as does the amount of base.

Once flocs are formed, the algae or cyanobacteria rapidly settle. This can be performed directly, or after culturing the algae briefly without carbon dioxide, to raise the pH so as to require less alkali. Depletion of carbon dioxide is assured by stopping the supply of carbon dioxide in the light, such that it is photosynthetically depleted. Flocculation can be achieved with many types of alkali, but slaked lime (calcium hydroxide) is preferable because of its low cost and preference as a cation in animal feed. As shown in the examples, it typically precipitates more cells than other basic flocculants. Slaked limestones or dolomites (mixtures of calcium and magnesium hydroxides) can also be used, to further reduce the cost from that of pure calcium hydroxide. In large-scale production the mixing of the alkali with the algae is performed just before entering the sedimentation tank using baffles to cause swirling and mixing. The cell flocs rapidly sediment and they can be removed in a continuous manner from the bottom of a vessel. Alternatively, dissolved air floatation could be used to float the flocs to the top of the vessel and the cells can be skimmed off. The cells can be further concentrated by low speed centrifugation and/or filtration technologies (including belt dryer, which would filtrate and dry), and then be processed by standard techniques.

Some algal species form extracellular polysaccharides that are of value for various uses, including as food thickeners, additives to quality papers or as a fermentation substrate. After cells are flocculated, these polysaccharide complexes can also be flocculated by adding more base, as described in the examples.

According to one embodiment of the present invention, all or part of the cell-free medium after flocculation is recycled back to culture facilities after the pH is lowered with carbon dioxide and phosphoric acids, needed for culture of marine micro-algae and cyanobacteria.

The results outlined in the examples belie all previous theories of flocculation, that there is a linear requirement for flocculant based on cell number, that flocculation by divalent ions bridge cells (monovalent ions are as good as divalent ions), that (insoluble) phosphate salt formation is part of the flocculation process (sodium, potassium and ammonium flocculate cells but do not form insoluble phosphate complexes), that magnesium gels must be formed, etc. Therefore the novel findings were rather novel and fully unpredicted.

The invention as outlined in FIG. 1 is now described by non-limiting examples. One of ordinary skill in the art would realize that various modifications can be made without departing from the spirit of the invention. The examples below show that the process according to this invention is useful, novel, non obvious and greatly simplifies the harvest and processing of microalgae and cyanobacteria.

In the various embodiments, algae and cyanobacteria were chosen from the following organisms: Phaeodactylum tricornutum, Amphiprora hyaline, Amphora spp., Chaetoceros muelleri, Navicula saprophila, Nitzschia sp., Nitzschia communis, Scenedesmus dimorphus, Scenedesmus obliquus, Tetraselmis suecica, Chlamydomonas reinhardtii, Chlorella vulgaris, Haematococcus pluvialis, Neochlorisoleo abundans, Synechococcus elongates PCC6301, Botryococcus braunii, Gloeobacter violaceus PCC 7421, Synechococcus PCC7002, Synechococcus PCC7942, Synechocystis PCC6803, Thermosynechococcus elongates BP-1, Nannochloropsis oculata, Nannochloropsis salina, Nannochloropsis spp., Nannochloropsis gaditana, Isochrysis aff. galbana, Aphanocapsa sp., Botryococcus sudeticus, Euglena gracilis, Nitzschia palea, Pleurochrysis carterae, Tetraselmis chuii, Pavlova spp. and Nannochloris spp. as representatives of all algae and cyanobacteria species. The algae come from a large taxonomical cross section of species (Table 1).

TABLE 1
Phylogeny of some of the eukaryotic algae used

| Genus | Family | Order | Phylum | Sub-Kingdom |
|---|---|---|---|---|
| Chlamydomonas | Chlamydomonadaceae | Volvocales | Chlorophyta | Viridaeplantae |
| Nannochloris | Coccomyxaceae | Chlorococcales | Chlorophyta | Viridaeplantae |
| Tetraselmis | Chlorodendraceae | Chlorodendrales | Chlorophyta | Viridaeplantae |
| Phaeodactylum | Phaeodactylaceae | Naviculales | Bacillariophyta | Chromobiota |
| Nannochloropsis | Monodopsidaceae | Eustigmatales | Heterokontophyta | Chromobiota |
| Pavlova | Pavlovaceae | Pavlovales | Haptophyta | Chromobiota |
| Isochrysis | Isochrysidaceae | Isochrysidales | Haptophyta | Chromobiota |

Note:
Many genes that in higher plants and Chlorophyta are encoded in the nucleus are encoded on the chloroplast genome (plastome) in the Chromobiota red lineage algae (Grzebyk, et al., 2003)

It is however, clear for one skilled in the art that this list is not exclusive, but that various other genera and species can be used as well.

### Example 1

### Algal and Cyanobacterial Flocculation

This example describes a method of algae harvest technique by induced cell flocculation.

### Materials and Methods

### Algal Cultivation:

Algae were cultured indoors in 2 L polyethylene (P.E) tubes. A constant temperature regime was maintained at 23° C., light:dark was set at 16:8 firs, light intensity of 100 μmol photons m⁻² s⁻¹. Marine species were cultured in filtered seawater; F/2 nutrient enrichment (Guillard and Ryther, 1962) was added every 72 hours at a dosage of 1:1000. Chlamydomonas reinhardtii was cultured in TAP culture medium (Gorman D. S. and R. P. Levine, 1965. P. Natl. Acad. Sci, USA 54: 1665-1669). Synechococcus 7942 was cultured in BG11 culture medium. Cultures were mixed by aeration. CO₂ was mixed with air and delivered to the cultures at controlled ratios via the aeration system.

### Time of Harvest:

Algae were harvested for experiments near their maximal culture densities.

### Method of Adding Alkali:

Alkali was added to 15 mL tubes (in triplicates) filled with 10 mL algae cell suspensions, in μL quantities. Calcium hydroxide was added as a fine suspension of particles in water (milk of lime) containing 0.15 g/mL Ca(OH)₂, NaOH and KOH were added as 0.15 g/mL solutions. NH₄OH was added as a 30% solution.

### Method of Measuring Flocculation pH:

pH was measured by a pH electrode (Pasco Scientific, Roseville, Calif.), using DataStudio software. Once flocs were visually observed, pH and quantity of base were recorded, and then calculated as μM.

### Method of Determining Flocculation:

Tubes were mixed following the addition of alkali. The onset of flocculation was characterized by a “grainy” appearance of the culture and was determined visually. To test the efficiency of pH induced flocculation and algae cell integrity, the protein content of a Nannochloropsis sp. culture grown indoors to a cell density of 2.46×10⁸ cells/mL was measured and compared to the protein content of the culture media, before and following flocculation treatments. Briefly, cells were centrifuged at 5,000 g, 15 minutes, 4° C.; triplicates were measured for each suspension volume. Supernatant was removed and the pellet was re-suspended in 1 mL sample buffer. 100 μl, glass beads were added and the cells were homogenized for 40 seconds using a bead beater (FastPrep™, MF). The homogenates were then centrifuged at 15,000 g, 15 minutes, 4° C. The resulting supernatant was transferred to a new vial and assayed using the Pierce® BCA Protein Assay Kit. Protein concentrations were calculated according to a standard curve. 10 mL aliquots of the above culture were treated with NaOH or Ca(OH)₂ in order to induce flocculation. Extra-cellular media protein concentration was assayed as described above, discarding the cell homogenization step.

### Results

TABLE 2
Flocculation of algae by non-toxic flocculants

| | NaOH | KOH | NH₄OH | Ca(OH)₂ | Fe(OH)₃ | Mg(OH)₂ |
|---|---|---|---|---|---|---|
| Species | pH of flocculation | | | | | |
| Chlamydomonas reinhardtii | | | | 7.97 | | |
| (wild type) | | | | | | |
| C. reinhardtii_(cell | | | | >12 Nf | | |
| wall-deficient) | | | | | | |
| Nannochloris | 10.2 | 10.2 | 10.1 | 10.0 | Nf | Nf |
| Tetraselmis | 9.55 | | | 9.65 | | |
| Phaeodactylum | 9.8 | 10.2 | 10.1 | 9.88 | Nf | Nf |
| Nannochloropsis | 9.45 | 9.5 | 9.64 | 9.5 | Nf | Nf |
| Pavlova | | | | 9.7 | | |
| Isochrysis | 8.8 | 9.7 | 9.7 | 8.65-9.3 | Nf | Nf |
| Synechococcus 7942 fresh water | | | | 9.7 | | |
| Synechococcus PCC 7002 | | | | 9.46 | | |
| marine | | | | | | |

Nf = no visible flocculation upon addition of 10% v/v flocculant solution; values are averages of two or more replicates. No determination was made where there are blank spaces.

TABLE 3
Flocculant molarity at flocculation

| | NaOH | KOH | NH₄OH | Ca(OH)₂ | Fe(OH)₃ | Mg(OH)₂ |
|---|---|---|---|---|---|---|
| Species | μmoles/L flocculant added at flocculation | | | | | |
| Chlamydomonas reinhardtii | | | | 6.07 | | |
| (wild type) | | | | | | |
| C. reinhardtii_(cell | | | | Nf | | |
| wall-deficient) | | | | | | |
| Nannochloris | 18.7 | 24.1 | 85.6 | 16.2 | Nf | Nf |
| Tetraselmis | | | | 10.5 | | |
| Phaeodactylum | 11.25 | 10.7 | 64.2 | 8.09 | Nf | Nf |
| Nannochloropsis | 11.3 | 12.5 | 28.5 | 9.2 | Nf | Nf |
| Pavlova | | | | 10.6 | | |
| Isochrysis | 10.0 | 9.8 | 49.9 | 5.06 | Nf | Nf |
| Synechococcus 7942 fresh water | | | | 4.04 | | |
| Synechococcus | | | | 6.3 | | |
| PCC 7002 marine | | | | | | |

Nf = no visible flocculation upon addition of 10% v/v flocculant solution; values are averages of two or more replicates. No determination was made where there are blank spaces.

Protein concentration in the algae media supernatant was below the detection level of the Pierce® BCA Protein Assay Kit after flocculation. Thus we deduced that pH-induced flocculation did not affect Nannochloropsis sp. cell integrity. The fact that the cell wall-deficient mutant of Chlamydomonas did not flocculate suggests that indeed it is cell wall determinants that govern the ability to flocculate. The fact that monovalent NaOH, NH₄OH, and KOH and divalent Ca(OH)₂ flocculate the algae at about the same molarities suggests that: 1. Only one charge of the calcium binds to the cell walls allowing them to flocculate; 2. that calcium is not bridging between cells and cross linking them in the manner that polyvalent polymers such as chitosan are thought to work; and 3. the mode of flocculation is not the previously proposed sweeping action of precipitated calcium and/or magnesium carbonates or phosphates, as ammonium, sodium, and potassium carbonates and phosphates are soluble, yet the cells flocculate at about the same molar values with mono and divalent ions.

TABLE 4
Efficacy of flocculation

| | NaOH | KOH | NH₄OH | Ca(OH)₂ | Fe(OH)₃ | Mg(OH)₂ |
|---|---|---|---|---|---|---|
| Species | % of cells remaining in supernatant | | | | | |
| Chlamydomonas | | | | 56 | | |
| reinhardtii | | | | | | |
| (wild type) | | | | | | |
| C. reinhardtii_(cell wall- | | | | 100 | | |
| deficient) | | | | | | |
| Nannochloris | 33.9 | 33.6 | 68.8 | 3.1 | Nf | Nf |
| Tetraselmis | | | | 5 | | |
| Phaeodactylum | 22.9 | 29.5 | 60.5 | 3.3 | Nf | Nf |
| Nannochloropsis | 56.1 | 63.3 | 22.3 | 4.5 | Nf | Nf |
| Pavlova | | | | 10 | | |
| Isochrysis | 20.4 | 4.6 | 5.0 | 10 | Nf | Nf |
| Synechococcus 7942 fresh water | | | | 5.7 | | |
| Synechococcus PCC 7002 | | | | 10 | | |
| marine | | | | | | |

Nf = no visible flocculation upon addition of 10% v/v flocculant solution; values are averages of two or more replicates.

### Example 2

### Effect of Culture Density on the Amount of Flocculant Needed to Induce Flocculation

The amount of flocculant needed to induce flocculation can affect the operating cost of algae harvesting systems. To this end we tested the effect of algal suspension density on the amount of Ca(OH)₂ needed to induce flocculation, demonstrating that flocculation was a function of the logarithm of the cell density and not a linear function of cell density, as had been previously thought.

### Materials and Methods

In order to test the relationship between algae density and the amount of flocculant needed to cause flocculation, cell suspensions cultured as described above were diluted with filtered seawater. Assays were run simultaneously on the initial and diluted suspensions. pH was measured and flocculation was induced and determined as described in Example 1.

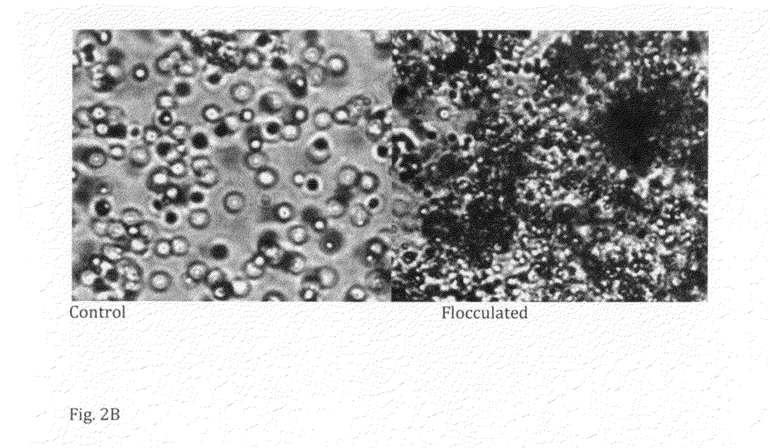

The results of this example are shown in FIG. 2. The 0.9 R² value presented in FIG. 2 indicates that the amount of Ca(OH)₂ needed to induce flocculation is a direct function of the logarithm of the algal cell density, and the amount of Ca(OH)₂ needed for algal flocculation at high densities is much less than would be predicted by the previously assumed linear function (see linear function line in FIG. 2), where it was considered that same number of positive charges per cell had to be titrated, irrespective of cell density, for flocculation to occur.

### Example 3

### Effect of Culture Density on the pH Value at the Onset of Flocculation

In order to compare our methodology with previously reported studies we tested the effect of algal suspension density on the pH value measured at flocculation.

### Materials and Methods

In order to test the effect of algae density on the pH value of culture media at the onset of flocculation (pH_{f}), cell suspensions cultured as describe above were diluted with filtered seawater. Assays were run simultaneously on the source and diluted suspensions. pH was measured by a pH electrode (Pasco Scientific, Roseville, Calif.) using DataStudio software. Flocculation was induced and determined as described above, at which point pH was recorded.

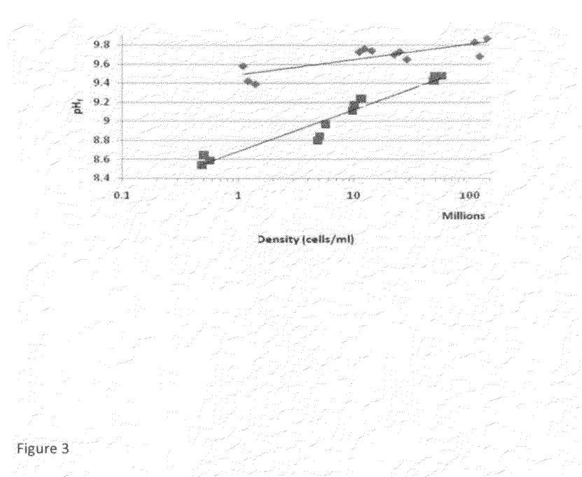

Results of this example are shown in FIG. 3.

The R squared values presented in FIG. 3 indicate that the pH_{f} value is a function of the logarithm of the cell density, but the actual effect is greater for Isochrysis sp. CS-177 than for Nannochloropsis salina. Therefore we assume that the unexpectedly low pH_{f} values we have observed may be dependent on cell density within the range of densities tested above.

### Example 4

### Effect of Culture and Initial pH on the pH Value of Flocculation (pH_{(f)})

The amount of flocculant needed to flocculate can affect the operating cost of algae harvesting systems. To this end we tested the effect of algal culture pH on the amount of Ca(OH)₂ needed to induce flocculation. pH at the time of harvest can be controlled by allowing cells to remove excess CO₂ from the medium by maintaining cultures in conditions (temperature, light, mixing, salinity) supporting photosynthesis while limiting CO₂ intake, resulting in an increase in medium pH.

### Materials and Methods

Polyethylene tubes containing 0.5 L of Nannochloropsis salina cultures were cultured for 7 days as described above, at two pH values, pH 7 (n=6 tubes) and pH 9 (n=3 tubes), allowing for several cell divisions throughout the experimental period. pH was maintained by controlling the CO₂ concentration in the aeration mixture. On day 8, CO₂ concentration in the aeration mixture of three of the pH 7 cultures was lowered for 3 hours, effectively allowing a rise in the culture pH to pH 8.35. Flocculation experiments using Ca(OH)₂ were conducted in duplicates on each treatment, as described above. Once flocs were observed, pH and quantity of base added were recorded.

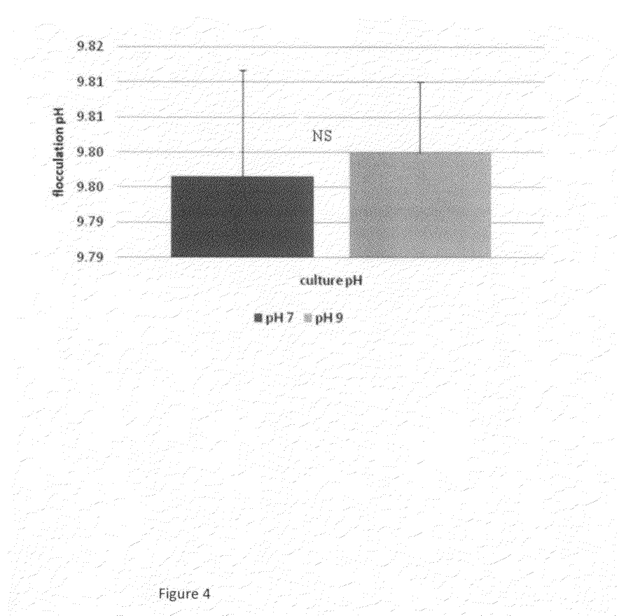

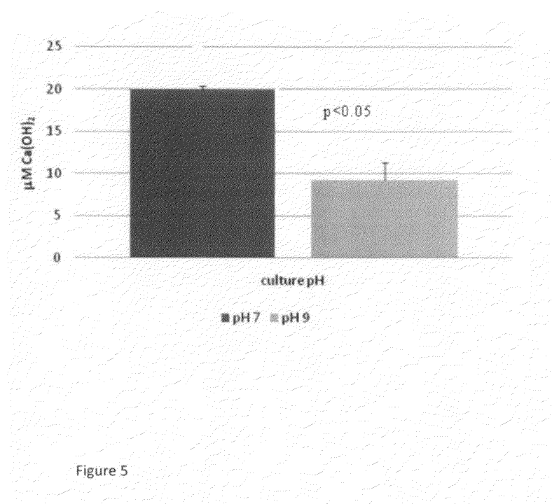

Results of this example are shown in FIGS. 4 and 5.

The results presented in FIG. 4 indicate that the pH value during the culture of Nannochloropsis salina does not affect the pH value at which these cells flocculate. Yet they indicate that by starving the cultures of carbon dioxide just prior to harvest, it is possible to halve the amount of Ca(OH)₂ needed to induce flocculation (FIG. 5).

### Example 5

### Effect of Culture Density on the Efficiency of Flocculation

In Example 1 it was demonstrated that unexpectedly, the amount of flocculant needed to induce flocculation was not linear with cell density but was a function of the logarithm of cell density. Those data did not deal with the actual efficiency of flocculation, namely what proportion of the cells were precipitated in the floc. Therefore, we tested the dependence of flocculation efficiency on culture density.

### Materials and Methods

Culture optical density at 750 nm (O.D_{(c)}) was measured using a spectrophotometer (Ultra spec 2100 pro, Amersham Biosciences), (n=9). Cell suspensions were diluted with filtered seawater and flocculation was induced with calcium hydroxide as described in Example 1. Flocs were allowed to settle and supernatants were decanted after 15 min. OD of the supernatant (OD_{(f)}) was measured at 750 nm and compared to the initial OD_{(c)}. Percent flocculation was described as 1−(OD_{(f)}/OD_{(c)}).

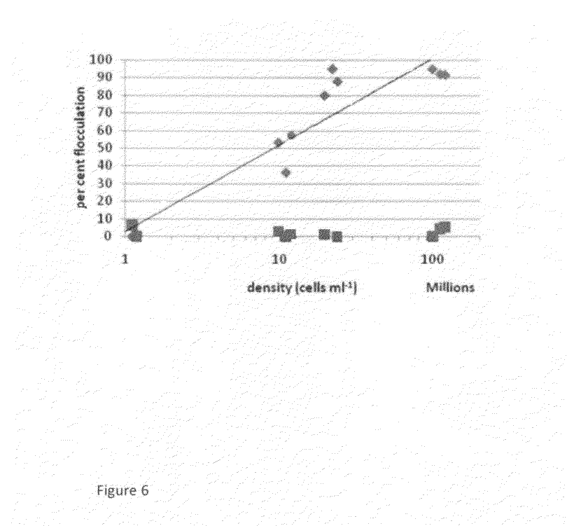

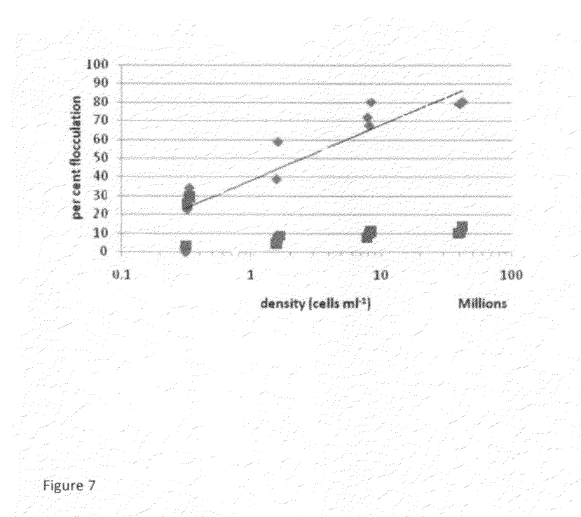

The results depicted in FIGS. 6, 7 imply that at the densities tested, ca. 90 percent of Nannochloropsis salina and ca. 80 percent of Isochrysis sp. CS-177 flocculation activity were a function of density at the time of flocculation. The efficiency flocculation increased with cell density.

### Example 6

### Effect of Culture Stage on Flocculation Parameters

The growth curve of algae cultures is similar to that of bacteria, including three distinct phases, lag, log and stationary. Physiological parameters of algae vary throughout the culture period. Their effect on the efficiency of flocculation may determine the desired timing of harvesting, by influencing the operating cost of algae harvesting systems. Therefore, we tested the dependency of flocculation efficacy on culture state. Only late logarithmic and stationary cells were tested, as in commercial practice they are the most likely to be harvested.

### Materials and Methods

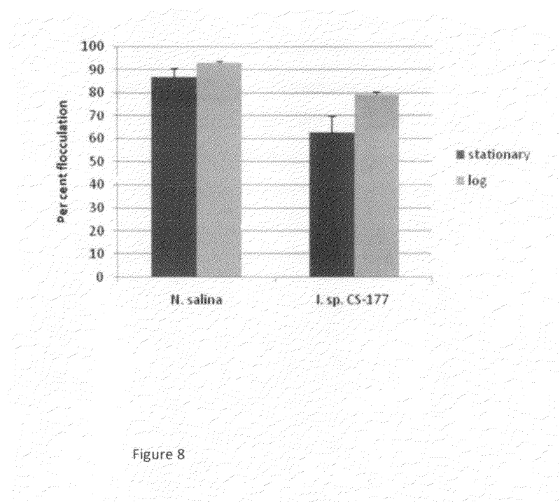

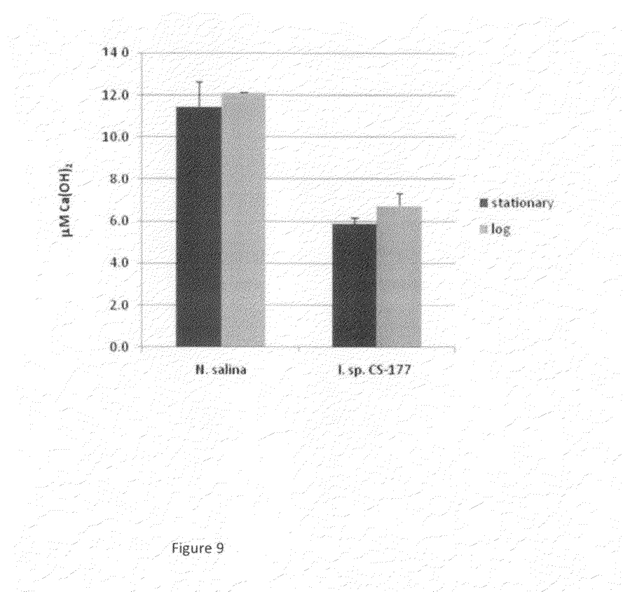

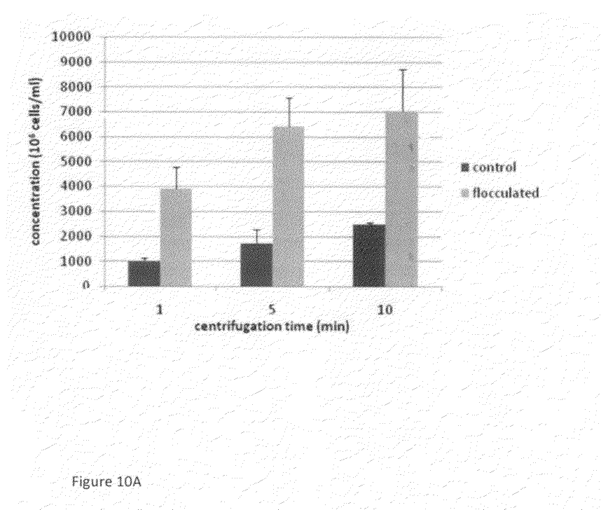

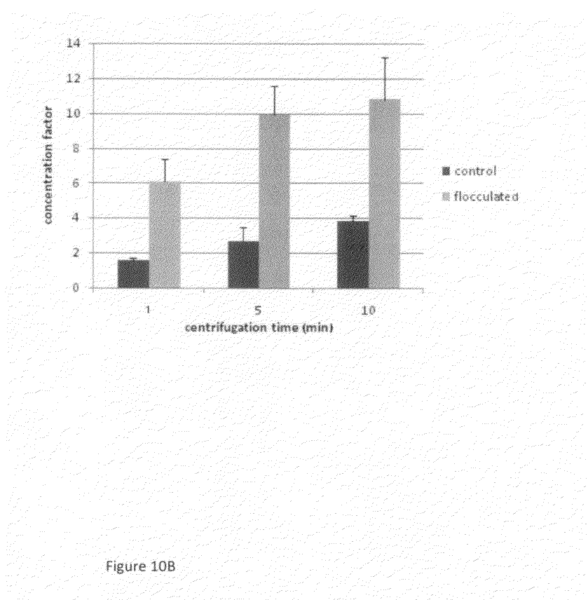

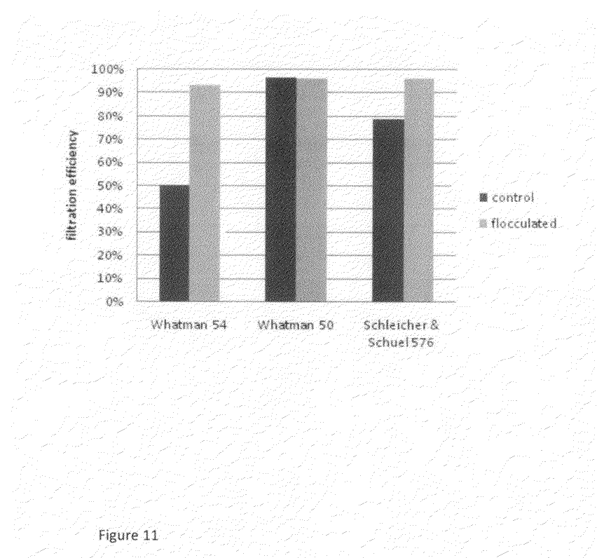

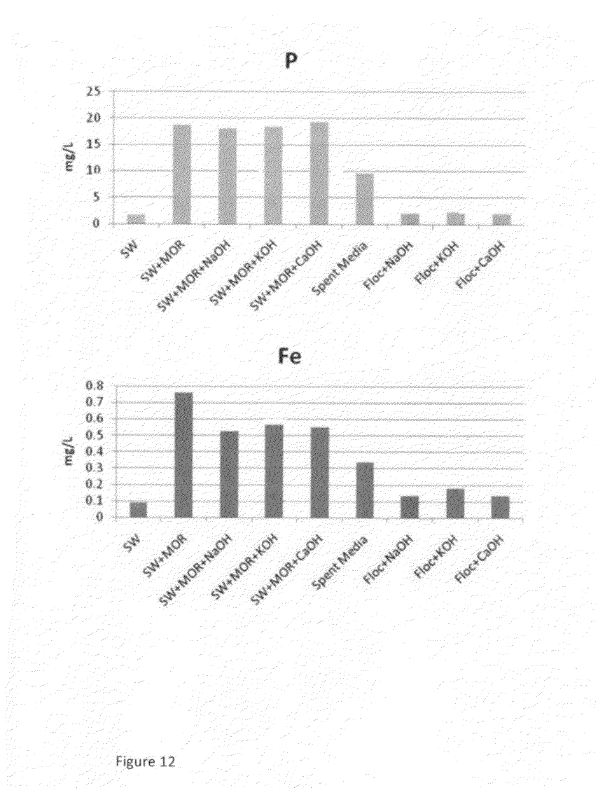

Cultures of Isochrysis sp. (n=3) and Nannochloropsis salina (n=3) were cultured as described above and counted daily to determine culture phase. Flocculation experiments using Ca(OH)₂ were conducted on each culture, at log and stationary phase, as described above. Once flocs were observed, pH and quantity of base added were recorded. Flocs were allowed to settle and supernatants were decanted after 15 min. OD of the supernatant (OD_{(f)}) was measured at 750 nm and compared to the initial OD_{(c)}. Percent flocculation was described as 1−(OD_{(f)}/OD_{(c)}). Results are shown in FIGS. 8 and 9.

Our results suggest that growth phase may affect the efficacy of flocculation of cultures (FIGS. 8, 9) in different directions. The differences in results are small, and while statistically significant may not be economically significant because of the opposing effects.

### Example 7

### Further Concentration of the Floc by Centrifugation

High speed (high g force) centrifugation is an effective but energy inefficient method for separating algal particles from an unflocculated suspension. The high capital as well as operating costs may rule out the use. Pre-concentration of particle mass and an increase in the average particle radius could reduce the mass of material that requires centrifugation and the force necessary to separate the suspended particles from the suspension, respectively. Our flocculation type of pre-centrifugation treatment process could lower the operating costs of centrifugation, because it should be possible to use much less costly lower g-force low speed centrifuges and reduce the time to sediment floc vs. cells. We tested the effect of algae suspension pre-concentration by flocculation as a pre-treatment for further concentration by centrifugation.

### Materials and Methods

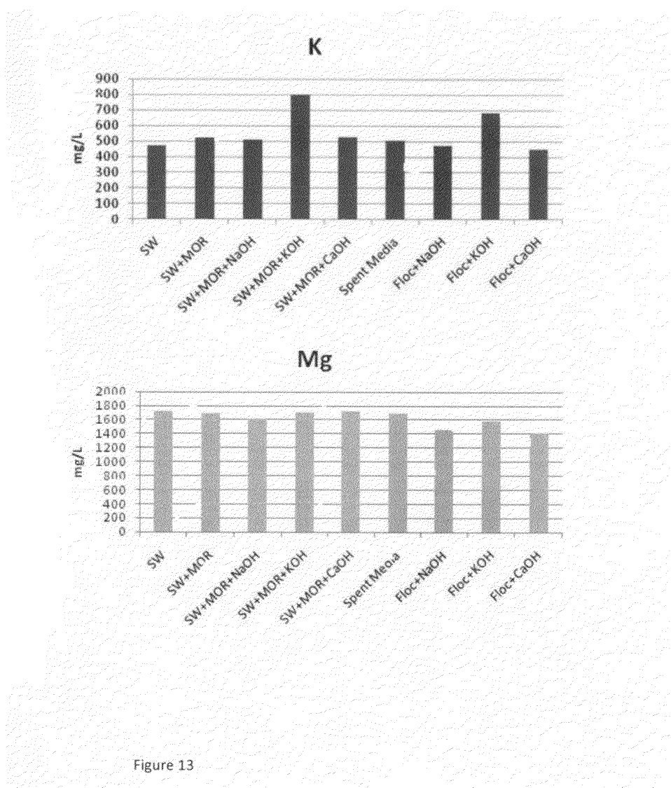

Three 1 L cultures of Nannochloropsis oculata at densities of 6.83×10⁸, 6.05×10⁸, and 6.56×10⁸ cells/mL, cultured as described above, were used. After measuring culture cell densities using a hemocytometer, six 10 mL aliquots were sampled from each culture, three were treated with Ca(OH)₂ to induce flocculation and three served as controls. Flocculated algae cells and controls were allowed to settle for 60 min whereupon supernatants (6.5 mL) were decanted from above the flocs and equivalent volumes were decanted from the control treatments. The remaining content was vortexed and 1.5 mL aliquots were transferred to micro-tubes. Treated samples and controls from each culture were centrifuged at 50×g for 1, 5 or 10 minutes. Supernatants were decanted and cell densities were measured using a hemocytometer. Concentration factor was calculated as: final cell concentration/initial cell concentration. Results of this example are shown in FIGS. 10A and B.

The results (FIGS. 10A and B) imply that flocculation as described above enables concentration of cells by a factor of 10 following centrifugation of 5 min at 50×g, 3.8 fold higher than prolonged sedimentation alone. No significant difference was found between 5 and 10 min centrifugation following flocculation. Since 65% of the culture volume may be discarded after flocculation and sedimentation, the process tested reduced the mass of material that entered the centrifugation process by 2.8 fold.

### Example 8

### Further Concentration of the Floc by Filtration

The effect of flocculation on the efficacy of filtration was tested on three different types of hardened filter papers using Nannochloropsis sp. cultured as described above.

Whatman grade 50 filters papers: retain particles as small as 2.7 μm, used for retention of very fine crystalline precipitates. The manufacturer states that it has a “slow flow rate, hardened and highly glazed surface. Suitable for qualitative or quantitative filtrations requiring vacuum assistance. They remain very strong when wet and will withstand wet handling and precipitate removal by scraping”.

Whatman grade 54 filter hardened papers “retain particles as small as 22 μm, and provide for very fast filtration. They are designed for use with coarse and gelatinous precipitates. Their high wet strength makes this grade very suitable for vacuum assisted fast filtration of ‘difficult’ coarse or gelatinous precipitates”.

### Materials and Methods

Culture optical density (OD_{(c)}) was measured at 750 nm using a spectrophotometer. Duplicate aliquots of 10 mL were filtered under vacuum pressure through the different filter papers. Optical density of the resulting filtrates (OD_{(f)}) was measured. Filtration efficiency was expressed as 1−(OD_{(f)}/OD_{(c)}). Cells were flocculated with calcium hydroxide, as in Example 7.

As is shown in FIG. 11, Whatman 50 paper removes both flocculated and suspended algal cells; Whatman 54 retains over 90% of the flocculated cells and removes 50% of the suspended cells.

### Example 9

### Possibility of Using Mixed Hydroxides of Calcium and Magnesium

Lime is produced by heating calcium carbonate to a high temperature, removing carbon dioxide, leaving CaO, which is then slaked with water to produce calcium hydroxide. Magnesium hydroxide is similarly produced from magnesium carbonate. Pure calcium carbonate and pure magnesium carbonate are far more expensive than various natural limestone/dolomite minerals containing various ratios of calcium and magnesium carbonates.

Thus, various artificial mixtures with different molar ratios of calcium hydroxide and magnesium hydroxide were prepared to ascertain whether less expensive mixtures could be used, and if there was an improvement of flocculation.

### Materials and Methods

Calcium hydroxide and magnesium hydroxide stock were prepared as 15% (w/v) suspensions in distilled water (DW). The effect of magnesium hydroxide on flocculation of algal species by calcium hydroxide was tested to ascertain if one synergistically improved the flocculation by the other (as claimed in the literature). This was done by adding a magnesium hydroxide suspension to a calcium hydroxide suspension at ratios of 1:3 and 1:1 and comparing them with pure calcium hydroxide. The suspensions were added to 15 mL tubes (in triplicates) filled with 10 mL algae cell suspensions, in μL quantities, as a fine suspension of particles in water. Flocculation pH, molarity of flocculant at flocculation and percent flocculation were measured.

### Method of Measuring Flocculation pH:

pH was measured by a pH electrode (Pasco Scientific, Roseville, Calif.), using DataStudio software. Once flocs were visually observed, pH and quantity of base were recorded as μM.

### Method of Determining Flocculation:

Tubes were mixed following the addition of alkali. Onset of flocculation was characterized by a “grainy” appearance of the culture and was determined visually.

### Results

TABLE 7
Flocculation of algae by distinct
[calcium:magnesium] ratios

| | [1:0] | [3:1] | [1:1] |
|---|---|---|---|
| Species | pH of flocculation | | |
| Nannochloris | 10.1 | 9.8 | 9.7 |
| Phaeodactylum | 10.0 | 9.8 | 9.5 |
| Nannochloropsis | 10.1 | 10.0 | 9.9 |

TABLE 8
Ca(OH)₂ molarity at flocculation at distinct
[calcium:magnesium] ratios

| | [1:0] | [3:1] | [1:1] |
|---|---|---|---|
| | μmoles Ca(OH)₂/L flocculant | | |
| Species | added at flocculation | | |
| Nannochloris | 12.1 | 9.1 | 6.1 |
| Phaeodactylum | 8.1 | 6.1 | 4.0 |
| Nannochloropsis | 10.1 | 7.6 | 5.1 |

TABLE 9
Effect of Mg(OH)₂ on Ca(OH)₂ flocculation efficacy at
distinct [calcium:magnesium] ratios

| | 1:0 | [3:1] | [1:1] |
|---|---|---|---|
| Species | % cells precipitated | | |
| Nannochloris | 26 | 22 | 22 |
| Phaeodadylum | 92 | 88 | 82 |
| Nannochloropsis | 55 | 84 | 68 |

Our results showed that flocculation pH (Table 7) and molarity of Ca(OH)₂ (Table 8) are reduced with addition of Mg(OH)₂ to a Ca(OH)₂ flocculant solution. Mg(OH)₂ showed a minor effect on flocculation efficacy in Phaeodactylum and Nannochloris However, it slightly improved flocculation efficacy of Nannochloropsis (Table 9). These results imply that hydroxides made by slaking various natural limestone/dolomite minerals containing various ratios of calcium and magnesium could serve as flocculants of certain algal species suspensions, potentially further lowering the cost of flocculation.

### Example 10

### Extracellular Polymer Precipitation from Spent Media Following Algal/Cyanobacterial Harvesting

This example describes the extraction of secreted, extracellular polymers such as polysaccharides, from spent culture media after major products (i.e. cell biomass) is harvested. Extracellular biological compounds secreted from algal/cyanobacterial cells during the different stages of culturing could potentially be valuable as commercial products. Secreted oils (lipids), proteins or carbohydrates as well as pigments or any other biological organic compounds are often found in large quantities in high-mass cultures. Extracting such compounds serves both the goal of maximizing profits from algal products as well as reducing costs for waste treatment required for disposal of high quantities of spent media. Additionally extracting biological compounds from spent media allows recycling of media for additional culture of algae, by reducing substrates for potential contaminants, thus reducing production costs.

### Materials and Methods

Algal culturing, time of harvest, method of flocculation, measuring flocculation pH and determining of flocculation efficiency are described in Example 1. Spent media, containing the remaining extracellular biological organic compounds, e.g. polysaccharides, as a non-limiting example, is further treated to feasibly extract such compounds.

Calcium hydroxide was added as a fine suspension of particles in water (milk of lime) containing 0.15 g/mL Ca(OH)₂ to triplicate of 1 L Isochrysis sp. cultures.

The cell pellet was harvested and the supernatant removed to a separation funnel. 20 mL of calcium hydroxide (milk of lime) containing 0.15 g/mL Ca(OH)₂ was then added as a fine suspension of particles in water. Two phases were formed in the funnel. The polysaccharide containing bottom phase was separated from the supernatant, pH and quantity of base [μM] were recorded, and the bottom phase was further concentrated by centrifugation at 1,000 g, 5 minutes, 4° C. Extracted polysaccharide volume was measured. Polysaccharide containing liquid was analyzed using an anthrone colorimetric assay (Gerhardt et al., 1994). Polysaccharide concentrations were calculated according to a standard curve. Additionally, total protein analysis (BCA™, Pierce, Rockford, USA) was performed on both the extracted substance and residual spent media (the suspension).

### Results

0.75 L of logarithmic growth phase Isochrysis sp CS-177 spent media was treated to extract polysaccharides. A sub-sample of 50 mL was saved prior to treatment and served as control. 50 mL of concentrated polysaccharides were ultimately extracted and analyzed. Carbohydrate content within Isochrysis culture was approx. 30%. Proteins (BCA™, Pierce, Rockford, USA) were not detected on both the extracted substance and residual spent media (the suspension). Polysaccharides were concentrated more than two-fold in the bottom phase.

### Example 11

### Composition of Process Media for Effluent Discharge

This example describes the potential of a flocculation process to additionally serve as a process enabling the discharge of seawater that has been used as growth media in algal mass culturing. In order to regulate discharge of media back to the environment local regulations must be followed to ensure that eutrophication does not occur i.e. the discharge of too much nutrients into the marine environment.

Another reason not to discharge nutrients is their cost. Nutrients are expensive resources for the growth process and recycling them lowers process costs thus improving existing practice. Therefore, we measured the levels of nutrients in the spent media following flocculation.

### Materials and Methods

### Algal Cultivation:

Algae were cultured outdoors in 2 L polyethylene (P.E) tubes. Maximal temperature was maintained around 27° C., using a fogger system. Marine species were cultured in filtered seawater; “Mor” nutrient enrichment (ICL Fertilizers, Table 10.) was added every 72 hours at a ratio of 1:1000.

TABLE 10
The composition of commercial fertilizer “Mor”, ICL Fertilizers

| N | P | K | Ca | Mg | Fe | |
|---|---|---|---|---|---|---|
| [%] | [%] | [%] | [%] | [%] | [ppm] | pH |
| 4 | 2.5 | 6% | 2 | 0.46 | 6 | 2.5 |

Cell densities of 6×10⁷ cultures were used for the process.

### ICP Analysis

Seawater (SW) was filtered through 0.2 μm membranes to remove microorganisms. Mor liquid mineral fertilizer (also filtered 0.2 μm) was added in relevant samples. Mor mineral fertilizer enriched seawater was titrated to pH=10 with each of the three flocculants. Spent media were prepared by centrifugation (4000×g, 10 min. 4° C.) and filtered 0.2 μm. Mor mineral fertilizer enriched cultures of Nannochloris were also titrated to pH=10 using Ca(OH)₂ with each of the three flocculants. The supernatant was filtered 0.2 μm. ARCOS, ICP Spectrometers, Spectro GMBH, Kleve, Germany was used for mineral analysis.

### Results

### Composition of Processed Media:

Addition of flocculant to Mor mineral fertilizer enriched seawater does not precipitate the phosphorous (P) (FIG. 12). This is direct evidence augmenting the indirect evidence describe in Example 1 that flocculation is not due to co-precipitation of calcium phosphate, as had been assumed in the literature. Some iron (Fe) from the mineral medium was co-precipitated by all three flocculants. The addition of flocculant to Mor mineral fertilizer enriched algae cultures reduce P and Fe concentrations to seawater values i.e. serving as a mechanism for eliminating nutrient discharge in the process effluent media.

### Example 12

### Composition of Processed Media for Re-Use

This example describes the potential of a flocculation process to serve additionally as a step enabling the reuse of sea water that has been used as growth media in algal mass culturing i.e. spent media after flocculation. In order to reduce operation costs in algal culturing i.e. pumping and treatment of raw seawater, the reuse of media after algae harvesting is essential. The composition of spent media was analyzed in order to assess the reuse of algae culture media after flocculation.

### Materials and Methods

### Algal Cultivation:

Algae were cultured outdoors in 2 L polyethylene tubes. Maximal temperature was maintained around 27° C., using a fogger system. Marine species were cultured in filtered seawater, “Mor” mineral fertilizer nutrient enrichment (ICL Fertilizers, Table 10.) was added every 72 hours at a ratio of 1:1000. Cell densities of 6*10⁷ cultures were used for the process.

ICP analysis is described in example 11.

### Results

### Composition of Process Media for Re-Use:

Addition of flocculant to Mor mineral fertilizer enriched SW did not affect concentrations of phosphorous (P) or magnesium (Mg) (FIG. 13). Iron (Fe) was slightly decreased (FIG. 12). Potassium (K) levels are unaffected by flocculation with NaOH or CaOH. KOH based flocculation naturally increased K concentrations. Addition of flocculant to Mor mineral fertilizer enriched algae cultures reduce P and Fe concentrations to seawater values while K and Mg concentrations were unaffected. These results imply that flocculation has no detrimental effect on dissolved nutrient concentrations thus signifying that flocculated media may be reused as culture media for algae.

### REFERENCES

Alfafara, C. G., Nakano, K., Nomura, N., Igarashi, T., Matsumura, M. (2002) Operating and scale-up factors for the electrolytic removal of algae from eutrophied lake water. Journal of Chemical Technology & Biotechnology 77: 8 871-876
Avnimelech, Y., Troeger, B. W., Reed, L. W. (1982) Mutual flocculation of algae and clay: evidence and implications. Science 216, 63-65.
Becker E W (1994) Microalgae biotechnology and microbiology. Cambridge Univ. Press. New York, N.Y.
Bernhardt, H. Clasen, J. (1994) Investigations into the flocculation mechanisms of small algal cells. nAqua—Journal of Water Supply: Research and Technology 43: 222-232.
Blanchemain, A; Grizeau, D (1999) Increased production of eicosapentaenoic acid by Skeletonema costatum cells after decantation at low temperature. Biotechnology Techniques 13: 497-501.
Bilanovic, D., Shelef, G. and Sukenik, A. (1988) Flocculation of microalgae with cationic polymers: effects of medium salinity. Biomass 17, 65-76.
Divakaran, R and V. N. S. Pillai (2002) Flocculation of algae using chitosan. J. Appl. Phycology 14:419-422
Dziubek, A. M., Kowal, A. L., 1989. High-pH coagulation-adsorption: a new technology for water treatment and reuse. Water Sci. Technol. 21, 511-517. 1962.
Elmaleh S., Coma J., Grasmick A., Bourgade L. (1991) Magnesium induced algal flocculation in a fluidized bed. Wat. Sci. Technol. 23, 1695-1702
Elmaleh S., H. Yahi, J. Coma (1996) Suspended solids abatement by pH. Increase—upgrading of an oxidation pond effluent. Wat. Res. 30, 2357-2362.
Folkman, Y., Wachs, A. M. (1973) Removal of algae from stabilization pond effluents by lime treatment. Water Research 7: 419-435.
Gerhardt, P., Murray, R G E., Wood, W A. Krieg, N R. (1994) Methods of general and molecular bacteriology. ASM, Washington D.C., ISBN 1-55581-048-9, p518.
Grzebyk, D., O, Schofield, P. Falkowski, J. Bernhard (2003) The Mesozoic radiation of eukaryotic algae: the portable plastid hypothesis. J. Phycol. 39:259-267.
Henderson, R. K., S. A. Parsons, B. Jefferson (2008a) Successful removal of algae through control of the zeta potential. Separation Sci. Tech. 43:1653-1666.
Henderson, R. K., S. A. Parsons, B. Jefferson (2008b) The impact of algal properties and pre-oxidation on solid-liquid separation of algae. Water Res. 42:1827-1845.
Henderson, R. K., S. A. Parsons, B. Jefferson (2008c) Surfactants as bubble surface modifiers in the flotation of algae: dissolved air flotation that utilizes a chemically modified bubble surface, Environ. Sci. Technol., 42: 4883-4888.
Knuckey, R. M., M. R. Brown, R. Robert, D. M. F. Frampton (2006) Production of microalgal concentrates by flocculation and their assessment as aquaculture feeds. Aquacultural Engineering 35:300-313.
Lavoie, A., de la Noue, J. (1983). Harvesting microalgae with chitosan. J. World Mariculture Soc., 14, 685 94.
Lee, S. J., S. -B. Kim, J. -E. Kiml, G. -S. Kwon, B. -D. Yoon, H. -M. Oh (1996) Effects of harvesting method and growth stage on the flocculation of the green alga Botryococcus braunii. Letters in Applied Microbiology 27: 14-18.
Leentvar, J., Rebhun, M., 1982. Effect of magnesium and calcium precipitation on coagulation—flocculation with lime. Water Res. 16, 655-662.
Levin, G. V., J. R. Clendenning, A. Gibor, F. D. Bogar (1962) Harvesting of algae by froth flotation. Appl. Microbiol. 10: 169-175.
McCausland, M. A., M. R. Braun, S. M. Barrett, J. A. Diemar, M. P. Heasman (1999) Evaluation of live microalgae and microbial pastes as supplementary food for Pacific oysters. Aquaculture 174:323-342.
Millamena, O. M., E. J. Aujero, I. G. Borlongan (1991) Techniques on algae harvesting and preservation for use in culture and a larval food. Aquacultural Eng. 9:295-304
Morales, J., de la Noiie, J., Picard, G. (1985). Harvesting marine microalgae species by chitosan flocculation. Aqua. Engng, 4, 257-70.
Nigam, B. P., Ramanathan, P. K., Venkataraman, L. V. (1980). Application of chitosan as a flocculant for the cultures of the green algae: Scenedesmus acutus. Arch. Hydrobiol., 88, 378-387.
Sayre; R. T. (2009) Optimization of biofuel production. United States Patent Application 20090181438
Semerjian, L., G. M. Ayoub (2003) High-pH-magnesium coagulation-flocculation in wastewater treatment. Advances in Environmental Research 7: 389-403.
Sukenik, A and G. Shelef (1984) Algal autoflocculation-verification and proposed mechanism. Biotechnology and Bioengineering, 26: 142-147.
Veloso, V., A. Reis, L. Gouveia, H. L. Fernandes, J. A. Empis, J. M. Novais (1991) Lipid production in Phaeodactylum tricornatum. Bioresource. Tech 38: 115-119.
Vrale, L., 1978. Chemical precipitation of wastewater with lime and seawater. Prog. Water Technol. 10, 645-656.
Yahi, H., S. Elmaleh, J. Coma (1994) Algal flocculation-sedimentation by pH increase in a continuous reactor. Water science & technology 30: 259-267, 1994
Zhang, G., B. Wang, P. Zhang, L. Wang, H. Wang (2006) Removal of algae by sonication-coagulation. Journal of Environmental Science and Health Part A, 41:1379-1390.

What is claimed is:

1. A method to harvest cyanobacteria or microalgae cells, said method comprising the steps of:
a) Culturing cyanobacteria or microalgae cells to a desired cell density of at least 5×10⁶ cells per ml;
b) Initiating flocculation by adding mono- or divalent alkaline flocculant or flocculants to a predetermined concentration, said predetermined concentration of the flocculant(s) being a function of logarithm of the cell density in culture;
c) Allowing the flocculation to complete; and
d) Harvesting flocculated cells.

2. The method according to claim 1, wherein the culture is deprived of carbon dioxide by stopping carbon dioxide supply in light before step b, whereby pH of the culture is increased and amount of alkaline flocculant needed in step b is decreased.

3. The method of claim 1, wherein the flocculant is selected from the group consisting of KOH, NaOH, NH₄OH, Ca(OH)₂, Mg(OH)₂, slaked and then hydrated limestone/dolomite minerals, or any mixture thereof.

4. The method of claim 2, wherein the flocculant is selected from the group consisting of KOH, NaOH, NH₄OH, Ca(OH)₂, Mg(OH)₂, slaked and then hydrated limestone/dolomite minerals, and any mixture thereof.

5. The method according to claim 3, wherein a step of centrifugation of less than 100×g, filtration, hydrodymanic separation in spiral separators or dissolved air flotation is added before or as part of step d.

6. The method according to claim 4, wherein a step of centrifugation of less than 100×g, filtration, hydrodymanic separation in spiral separators or dissolved air flotation is added before or as part of step d.

7. The method of claim 1, wherein solution remaining after harvesting is recycled back to the culture bioreactor.

8. The method of claim 3, wherein solution remaining after harvesting is recycled back to the culture bioreactor.

9. The method of claim 4, wherein solution remaining after harvesting is recycled back to the culture bioreactor.

10. The method of claim 1, wherein the cultured algal or cyanobacterial species is selected from the group consisting of Phaeodactylum tricornutum, Amphiprora hyaline, Amphora spp., Chaetoceros muelleri, Navicula saprophila, Nitzschia sp., Nitzschia communis, Scenedesmus dimorphus, Scenedesmus obliquus, Tetraselmis suecica, Chlamydomonas reinhardtii, Chlorella vulgaris, Haematococcus pluvialis, Neochloris oleo abundans, Synechococcus elongatus PCC6301, Botryococcus braunii, Gloeobacter violaceus PCC7421, Synechococcus PCC7002, Synechococcus PCC7942, Synechocystis PCC6803, Thermosynechococcus elongatus BP-1, Nannochloropsis oculata, Nannochloropsis salina, Nannochloropsis spp., Nannochloropsis gaditana, Isochrysis all galbana, Aphanocapsa sp., Botryococcus sudeticus, Euglena gracilis, Nitzschia palea, Pleurochrysis carterae, Tetraselmis chuii, Pavlova spp. and Nannochloris spp.

11. A method to increase efficiency of harvesting algae or cyanobacteria by low speed centrifugation, filtration, dissolved air flotation, or by hydrodynamic separation in spiral separators, said method comprising the steps of:
a. Culturing cyanobacteria or microalgae cells to a desired cell density of at least 5×10⁶ cells per ml;
b. optionally pre-concentrating cultured cells to obtain the desired cell density of at least 5×10⁶ cells per ml using hydrodynamic separation in spiral separators;
c. Initiating flocculation by adding alkaline flocculant to a predetermined concentration, said predetermined concentration of the flocculant being a function of logarithm of the cell density in culture;
d. Allowing the flocculation to complete;
e. Applying centrifuge of less than 100×g, filtration, dissolved air flotation, or hydrodynamic separation in spiral separators to flocculated cells to further dewater; and
f. harvest flocculated cells.

12. The method of claim 15, wherein suspension remaining after harvesting is recycled back to the culture bioreactor.
